(19) United States
(12) Patent Application Publication
(10) Pub. No.: US 2009/0248084 A1
(43) Pub. Date: Oct. 1, 2009

(54) SYSTEMS AND METHODS FOR PERFORMING ANKLE ARTHRODESIS IN A HUMAN PATIENT

(76) Inventor: Beat Hintermann, Liestal (CH)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/416,530

(22) Filed: Apr. 1, 2009

Related U.S. Application Data
(63) Continuation of application No. PCT/FR2008/000424, filed on Mar. 27, 2008.
(60) Provisional application No. 61/072,614, filed on Apr. 1, 2008.

(51) Int. Cl. A61B 17/80; A61B 17/88

(52) U.S. Cl. 606/286; 606/280; 606/281

(57) ABSTRACT

The present disclosure relates to systems and methods for ankle arthrodesis where the joint between the tibia and the talus of a human patient can be immobilzed. The system in one aspect can comprise an antero-lateral plate and an antero-medial plate.

### RELATED APPLICATIONS

This application is a continuation of PCT International Patent Application No. PCT/FR2008/000424, filed Mar. 27, 2008 and claims the benefit of U.S. Provisional Patent Application No. 61/072,614, filed Apr. 1, 2008, the disclosures of which are incorporated herein by reference in their entirety.

### TECHNICAL FIELD

The subject matter disclosed herein relates generally to surgical devices and methods designed to secure together two bone portions of a joint, e.g. with a view to performing arthrodesis. More particularly, the subject matter disclosed herein relates to systems and methods for immobilizing the joint between the tibia and the talus of a human patient.

### BACKGROUND

In certain pathologies that occur at the joint between the tibia and the talus (primary osteoarthritis, post-traumatic osteoarthritis, or ankle arthritis) or after failure of total ankle replacement, it can be necessary to perform arthrodesis in order to fuse the two bones (talus and tibia) together. In general arthrodesis operations are delicate because they lock a joint irreversibly in a defined position. Arthrodesis of the joint between the tibia and the talus is highly important insofar as that joint plays an essential part in the walking cycle of a human being. It can thus be understood that it is essential, in particular in view of the load cycles to which the joint between the tibia and the talus is subjected, that the arthrodesis of that joint be performed in the least invasive and most precise manner possible, so as to avoid any subsequent discomfort.

Currently, ankle arthrodesis is usually performed by means of bone fastener plates provided with holes designed to receive fastener screws for securing the plate to the bone to be fused.

Plates are thus known that are designed to be fastened to the lateral side of the tibia or of the talus. Unfortunately, those plates are generally difficult to fit in particular because of the operating difficulty caused by the presence of the fibula. In addition, those known plates have a shape that does not enable them fit snugly over the bone outlines, which gives rise to a risk of subsequent discomfort and complications for the patient, and does not facilitate surgical intervention. In order to remedy that problem, the surgeon can be obliged to perform steps of shaping the plate during the operation, by folding it manually (using pliers). Such folding steps constitute a significant additional constraint in surgical intervention, and can also weaken the metal at the fold zone, thereby constituting a major drawback in terms of strength. In addition, the need to provide openings for allowing the fastener screws to pass through the plate complicates the plate-folding step precisely because of the presence of the openings.

Thus, known plates do not make it possible to perform tibia/talus arthrodesis under good operating conditions, and with minimized risk of subsequent discomfort and complications for the patient.

### SUMMARY

An object of the present disclosure is therefore to remedy the various above-listed drawbacks and to provide novel systems and methods for immobilizing the joint between the tibia and the talus of a human patient that makes it possible to perform arthrodesis under good operating conditions while also making it possible to obtain bone fusion that is particularly stable, strong, precise, and comfortable for the patient.

Another object of the present disclosure is to provide novel systems and methods for immobilizing the joint between the tibia and the talus of a human patient that is particularly comfortable for the patient, under all circumstances.

Another object of the present disclosure is to provide novel systems and methods for immobilizing the joint between the tibia and the talus of a human patient that is particularly easy and quick to put in place, and that procures particularly reliable bone fusion.

Another object of the present disclosure is to provide novel systems and methods for immobilizing the joint between the tibia and the talus of a human patient that facilitates the process of osteosynthesis between the tibia and the talus.

Another object of the present disclosure is to provide novel systems and methods for immobilizing the joint between the tibia and the talus of a human patient that makes it possible to facilitate osteosynthesis in a manner that is particularly simple, quick, and reliable.

The objects assigned to the present disclosure are achieved by systems and methods as described herein for immobilizing the joint between the tibia and the talus of a human patient. The systems and methods of the present disclosure can comprise: an antero-lateral plate that itself can comprise a tibio-lateral strip and a talo-lateral tab interconnected by a link zone. The tibio-lateral strip, the talo-lateral tab, and the link zone can be shaped so that at least one portion of the tibio-lateral strip can rest substantially against the anterior lateral face of the tibia that is situated towards the outside of the body of the patient relative to the anterior tibial crest. The tibio-lateral strip, the talo-lateral tab, and the link zone can also be shaped so that the talo-lateral tab can rest substantially against the talus. An antero-medial plate can also be provided that itself can comprise a tibio-medial strip and a talo-medial tab interconnected by a link zone. The tibio-medial strip, the talo-medial tab, and the link zone can be shaped so that: at least one portion of the tibio-medial strip can rest substantially against the anterior medial face of the tibia that is situated towards the inside of the body of the patient relative to the anterior tibial crest and also so that the talo-medial tab can rest substantially against the talus.

These and other objects are achieved by the systems and methods disclosed herein.

### BRIEF DESCRIPTION OF THE DRAWINGS

Other advantages and objects of the present disclosure appear in more detail on reading the following description and on examining the accompanying drawings that are given merely by way of explanatory and non-limiting example, and in which:

FIG. 1 is a front view (ventral view) of the antero-lateral and antero-medial plates of a system of the present disclosure, as positioned on the right tibia and the right talus of a human patient so as to immobilize the joint between the tibia and the talus;

FIG. 2 is identical to FIG. 1, except that it is a lateral side view rather than a front view (ventral view);

FIG. 3 is identical to FIGS. 1 and 2 except that it is a medial side view;

FIG. 4 is a lateral side view of the antero-lateral plate of FIGS. 1 to 3;

FIG. 5 is a medial side view of the antero-lateral plate of FIG. 4;

FIG. 6 is a rear view (dorsal view) of the antero-lateral plate of FIGS. 4 and 5;

FIG. 7 is a front view (ventral view) of the antero-lateral plate of FIGS. 4 to 6;

FIG. 8 is a front view (ventral view) of the antero-medial plate of FIGS. 1 to 3;

FIG. 9 is a rear profile view of the antero-medial plate of FIG. 8;

FIG. 10 is a lateral view of the antero-medial plate of FIGS. 8 and 9;

FIG. 11 is a medial view of the antero-medial plate of FIGS. 8 and 10;

FIG. 12 is a rear view (dorsal view) of the antero-medial plate of FIGS. 8 to 11;

FIG. 13 shows a pair of plates made up of an antero-lateral plate and of an antero-medial plate that are part of a system of the present disclosure for immobilizing the joint between the right tibia and the right talus of a human patient;

FIG. 14 shows a front view of a pair of plates made up of an antero-lateral plate and of an antero-medial plate that are part of a system of the present disclosure and that are designed to immobilize the joint between the left tibia and the left talus of a human patient;

FIG. 15 is a side view showing the antero-lateral plate of FIG. 14 co-operating with bone anchor screws;

FIG. 16 is a plan view of pliers for compression of the joint between the tibia and the talus.

FIG. 17 is a perspective view of a fastener and guide bush designed to be associated with a first jaw of the pliers of FIG. 16;

FIG. 18 is a perspective view of a fastener screw designed to be associated with the second jaw of pliers of FIG. 16;

FIG. 19 shows how the pliers of FIG. 16 co-operate firstly with the bush of FIG. 17 and secondly with the screw of FIG. 18;

FIG. 20 shows how the compression tool of FIG. 19 are implemented in co-operation with the antero-medial plate of FIG. 14;

FIGS. 21 and 22 show two examples of screws (with heads that are respectively frustoconical and spherical) and of corresponding lock screws (systems of the SURFIX® type) suitable for fastening the antero-lateral and antero-medial plates to the bone.

### DETAILED DESCRIPTION

The present disclosure relates to systems and methods for immobilizing the joint between the tibia 1 and the talus 2 of a human patient, with a view to performing arthrodesis of the ankle. The systems and methods of the present disclosure thus advantageously constitute ankle arthrodesis systems and methods. The systems and methods described and shown herein can include at least an antero-lateral plate 3 designed to be fastened both to the anterior (ventral) face of the tibia 1 and to the anterior face of the talus 2. The antero-lateral plate 3 is a plate that can be implanted surgically. Antero-lateral plate 3 can be rigid, and it can be made of a hard and rigid material such as stainless steel, titanium, or any other suitable material, such as any suitable metal material of surgical grade.

The antero-lateral plate 3 itself can comprise a tibio-lateral strip and a talo-lateral tab 5 that can be interconnected by a link zone 6. In other words, the tibio-lateral strip 4 can be extended by the link zone 6, which itself can be extended by the talo-lateral tab 5. The antero-lateral plate 3 can have an elongate overall shape, as shown in the figures, i.e. the overall length of the antero-lateral plate 3 can be significantly greater both than its width and than its thickness.

In accordance with the present disclosure, the tibio-lateral strip 4, the talo-lateral tab 5, and the link zone 6 can be shaped both so that: at least one portion of the tibio-lateral strip 4 can rest substantially against the anterior lateral face 1A of the tibia 1 that is situated towards the outside of the body of the patient relative to the anterior tibial crest 1B; and also so that the talo-lateral tab 5 can rest substantially against the talus 2, and preferably against the neck 2A of the talus 2. In other words, the tibio-lateral strip 4, the talo-lateral tab 5, and the link zone 6 can be positioned, oriented, and dimensioned relative to one another so that when the antero-lateral plate 3 is installed in its final implantation position, inside the body of the patient, the tibio-lateral strip 4 can substantially snugly fit the anterior lateral face 1A of the tibia 1, while the talo-lateral tab 5 can be substantially pressed against the talus 2, and preferably against the neck 2A of the talus 2. The antero-lateral plate 3 thus can be designed to be positioned in register with the distal end of the tibia 1, and to be pressed against the lateral anterior face 1A thereof.

Advantageously, as shown in the figures, the antero-lateral plate 3 can be shaped so that, when it finds itself in its final implantation position, its tibio-lateral strip 4 extends substantially against the anterior tibial crest 1B, and parallel thereto. The tibio-lateral strip 4 thus can be pressed against the portion of the anterior lateral face 1A that is in the immediate vicinity of the anterior tibial crest 1B, and the portion in question of the anterior lateral face 1A can be substantially flat, which makes it possible for the tibio-lateral strip 4 to be fastened stably and effectively to the tibia 1, as described herein.

The link zone 6 of the antero-lateral plate 3 can come into register with at least a portion of the tibial pilon 1C, which can form a bulge of bone at the distal end of the tibia 1. In order to make the antero-lateral plate 3 easier for the patient to tolerate and thus more comfortable for the patient, the link zone 6 of the antero-lateral plate 3 can have a shape that can be substantially complementary to the shape of the portion of the tibial pilon 1C that it covers.

The link zone 6 of the antero-lateral plate 3 can have a shape that can be curved significantly so as to match the outline of the tibial pilon 1C, as shown in the figures. Thus, the antero-lateral plate 3 of the present disclosure can be anatomical in general shape, i.e. it can be shaped to match as closely as possible the anatomy of the bone structures to which it is designed to be fastened. By this feature, the antero-lateral plate 3 is easier for the patient to tolerate and easier for the surgeon to fit.

Surgically putting the antero-lateral plate 3 into place is also greatly facilitated by the specific positioning chosen, i.e. the positioning on the anterior lateral face 1A of the tibia 1. This positioning facilitates the work of the surgeon because it requires only an anterior approach.

Such an anterior approach procures better visibility than lateral, posterior, or medial approaches, and it requires only a short incision in comparison with the incisions that are required for the other above-mentioned approaches, and fitting the plate is facilitated because the anterior approach is not obstructed by anatomical obstacles such as the fibula (an obstacle for the lateral approach), the Achilles tendon (an obstacle for the posterior approach), or the malleolus (an obstacle for the medial approach). In addition, it is already known that the anterior surgical approach can be used for fitting total ankle prostheses. It is thus very advantageous to propose for the same surgeons to use an anterior approach for performing talo-tibial arthrodesis, in particular since the arthrodesis can take place after fitting a total ankle prosthesis has failed. The present disclosure has identified advantageous anatomical positioning for the antero-lateral plate 3 and can provide an advantageous geometrical shape for the antero-lateral plate 3, which makes it possible to match as closely as possible the anatomy of the particular implantation site, thereby procuring fastening that is reliable, effective, and comfortable.

The systems and methods of the present disclosure further can comprise an antero-medial plate 7. The antero-medial plate 7 is a plate that is also can be implanted surgically, preferably via the same anterior approach as the antero-lateral plate 3. The antero-medial plate 7 also can be rigid and is also can be made of a hard and rigid material such as stainless steel, titanium, or any other suitable material, such as any other suitable metal material of a surgical grade.

The antero-medial plate 7 itself can comprise a tibio-medial strip 8 and a talo-medial tab 9 that can be interconnected by a link zone 10. In other words, the tibio-medial strip 8 can be extended by the link zone 10, which itself can be extended by the talo-medial tab 9. The antero-medial plate 7 can have an elongate overall shape, i.e. its overall length can be significantly greater both than its width and than its thickness.

The tibio-medial strip 8, the talo-medial tab 9, and the link zone 10 of the antero-medial plate 7 can be shaped both so that: at least one portion of the tibio-medial strip 8 can rest substantially against the anterior medial face 1D of the tibia 1 that is situated towards the inside of the body of the patient relative to the anterior tibial crest 1B; and also so that the talo-medial tab 9 can rest substantially against the talus 2, and can rest against the neck 2A of the talus 2.

Thus, one advantageous feature of the systems and methods of the present disclosure lies in the implementation of two plates 3, 7 that in one aspect can be disposed towards the distal portion of the tibia 1, at least partially on either side of the anterior tibial crest 1B, the antero-lateral plate 3 and the antero-medial plate 7 being designed to be secured both to the distal portion of the tibia 1 and to the talus 2. Such an arrangement procures fastening that is particularly stable and effective, while also being particularly well tolerated by the patient because the antero-medial plate 7, like the antero-lateral plate 3, matches as closely as possible the outline of the bone structure against which it is mounted and to which it is designed to be fastened.

The systems and methods of the present disclosure are particularly easy to implement or fit because the antero-medial plate 7, like the antero-lateral plate 3, can be fitted via the anterior approach, with the above-described ensuing advantages. Both plates, i.e. the antero-lateral plate 3 and the antero-medial plate 7, can also be fitted via the same anterior incision.

The link zone 10 of the antero-medial plate 7 can be designed or configured to come into register with at least a portion of the tibial pilon 1C, the link zone 10 of the antero-medial plate 7 having, for this purpose, a shape that can be substantially complementary to the shape of the portion of the tibial pilon 1C.

The antero-medial plate 7 thus advantageously can snugly fit the outline of the anatomical structures that it covers and to which it is designed to be secured, thereby contributing not only to the reliability and to the stability of the fastening, but also to the comfort of the patient.

Advantageously, and as shown in the figures, the antero-medial plate 7 can be shaped so that, when it finds itself in its final implantation position, its tibio-medial strip 8 can extend at least in part substantially against the anterior tibial crest 1B, and parallel thereto, on the medial side. The tibio-medial strip 8 thus can at least in part be pressed against the portion of the anterior medial face 1D that is in the immediate vicinity of the anterior tibial crest 1B, and the portion in question of the anterior medial face 1D can be substantially flat, which makes it possible for the tibio-medial strip 8 to be fastened stably and effectively to the tibia 1, as described herein.

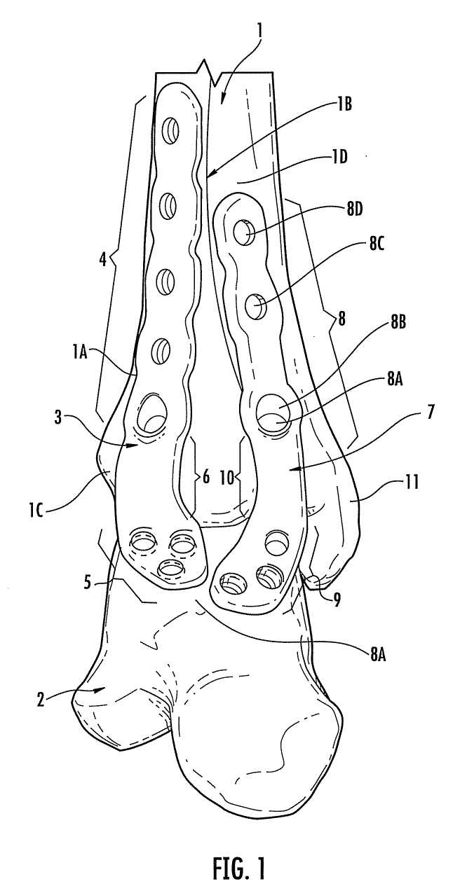

More precisely, and as shown in the figures, the antero-medial plate 7 can be designed or configured to be positioned in part straddling the anterior tibial crest 1B. As is well known, and as shown in the figures, the anterior tibial crest 1B is not rectilinear over the entire length of the tibia 1, and it is inclined towards the internal malleolus 11 in the vicinity of the distal end of the tibia 1. The antero-medial plate 7 can thus advantageously be designed so that only an end portion of its tibio-medial strip 8 that is situated towards the free end thereof is disposed medially relative to the anterior tibial crest 1B. In other words, the antero-medial plate 7 can be designed so that its talo-medial tab 9 can substantially snugly fit the neck 2A of the talus 2 and can be extended by the link zone 10 can snugly fit most of the bulge formed by the tibial pilon 1C. The link zone 10 can then be extended by a first portion (or distal portion) of the tibio-medial strip 8, the first portion being situated under the anterior tibial crest 1B, the distal portion itself being extended by a second portion (or end portion) which can extend substantially parallel to and against the anterior tibial crest 1B (see FIG. 1).

The present disclosure has identified advantageous anatomical positioning for the antero-medial plate 7, and provides an advantageous geometrical shape for the antero-medial plate 7, which makes it possible to match as closely as possible the anatomy of the particular implantation site, thereby procuring fastening that is reliable, effective, and comfortable.

Advantageously, and as can be seen in particular in FIGS. 9 and 10, the antero-medial plate 7 can in one aspect have a significantly twisted shape so as to match both the outline of the anterior medial face 1D of the tibia 1 and also the outline of the talus 2 and more precisely of the neck 2A of the talus 2. The antero-lateral plate 3 also can have such a significantly twisted shape, so as to match both the outline of the anterior side face 1A and the outline of the neck 2A of the talus 2.

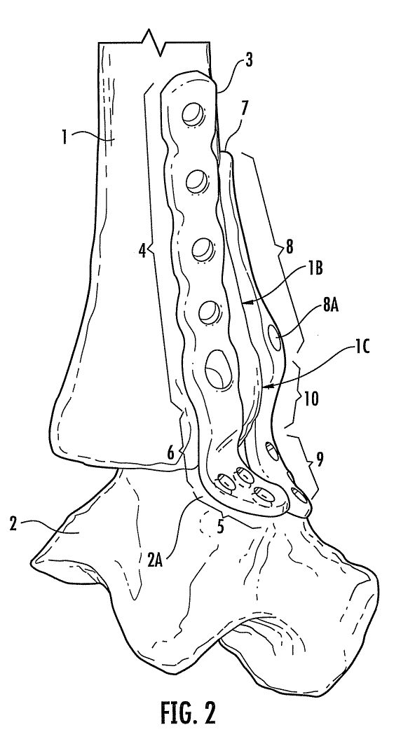

Advantageously, the tibio-medial strip 8 of the antero-medial plate 7 can have a substantially arcuate shape, as can be seen in particular in FIGS. 2, 3, and 10, so as to match both the outline of the anterior medial face 1D of the tibia 1 and also the outline of at least a portion of the tibial pilon 1C. In other words, the tibio-medial strip 8 can be shaped and dimensioned so as not only to extend over the anterior medial face 1D, but also to fit snugly over the beginning of the bulge formed by the pilon 1C, the remainder of the bulge fitting snugly into the link zone 10, which can be extended by the talo-medial tab 9.

By the particular positioning of the antero-lateral plate 3 and of the antero-medial plate 7 as identified herein, it is possible, with only two plates that are small in size and easy to fit, to obtain fastening that is extremely robust, stable, and comfortable, and that is, in addition, easy for the surgeon to perform, in particular in view of the techniques known from the prior art.

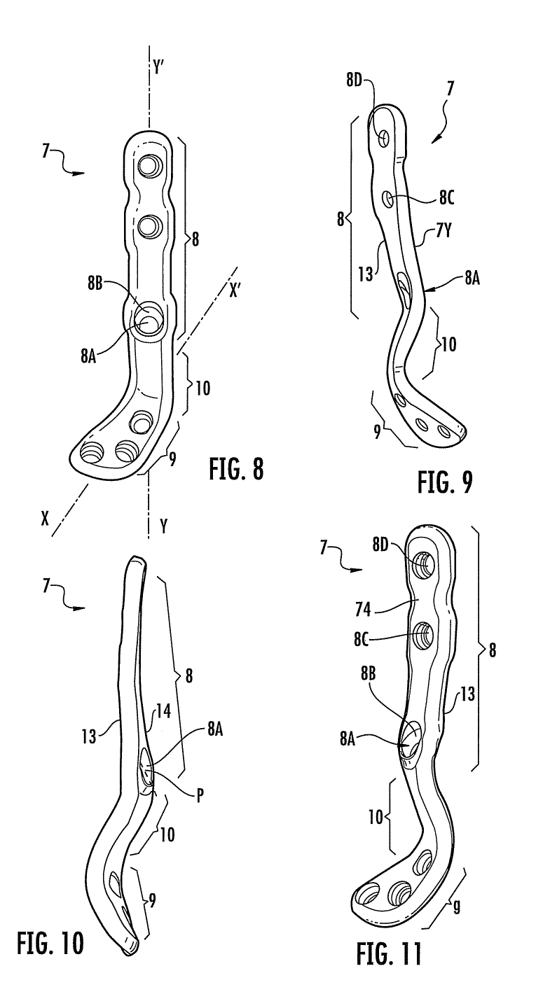

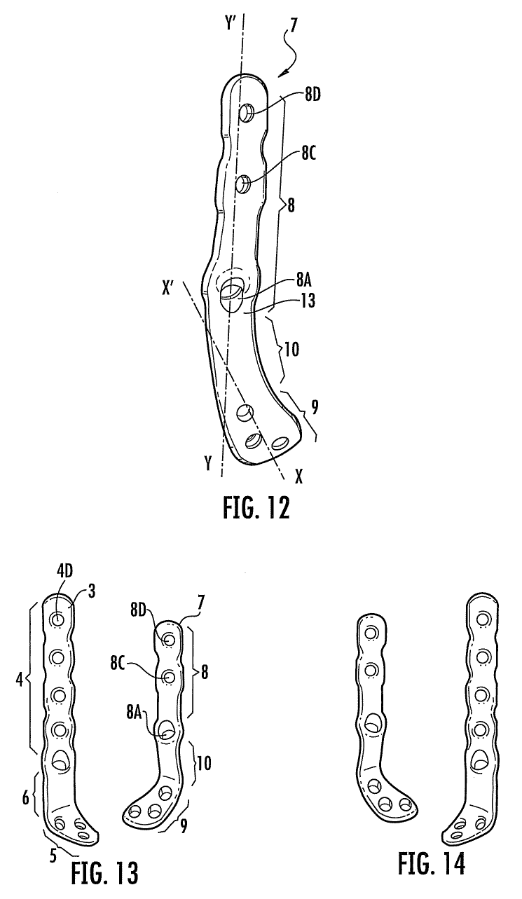

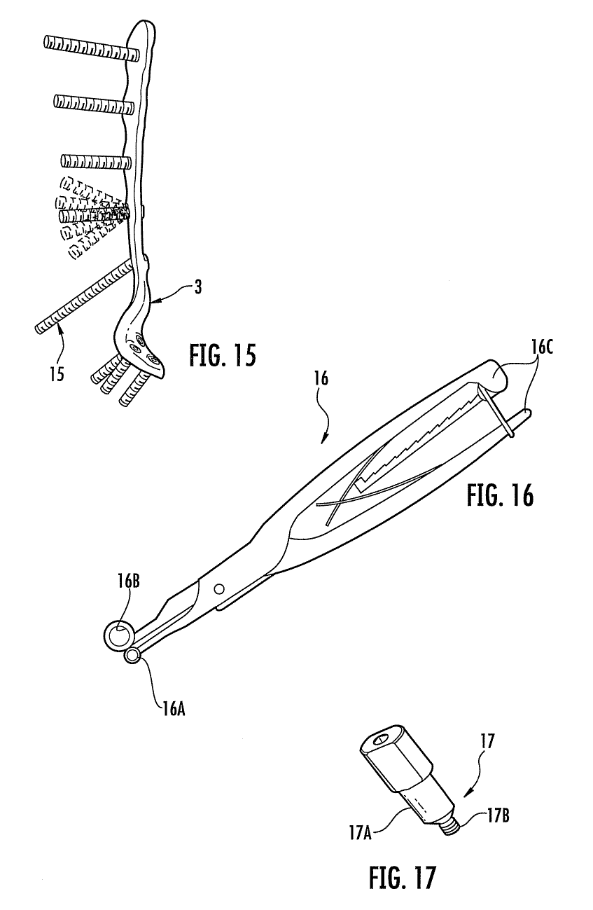

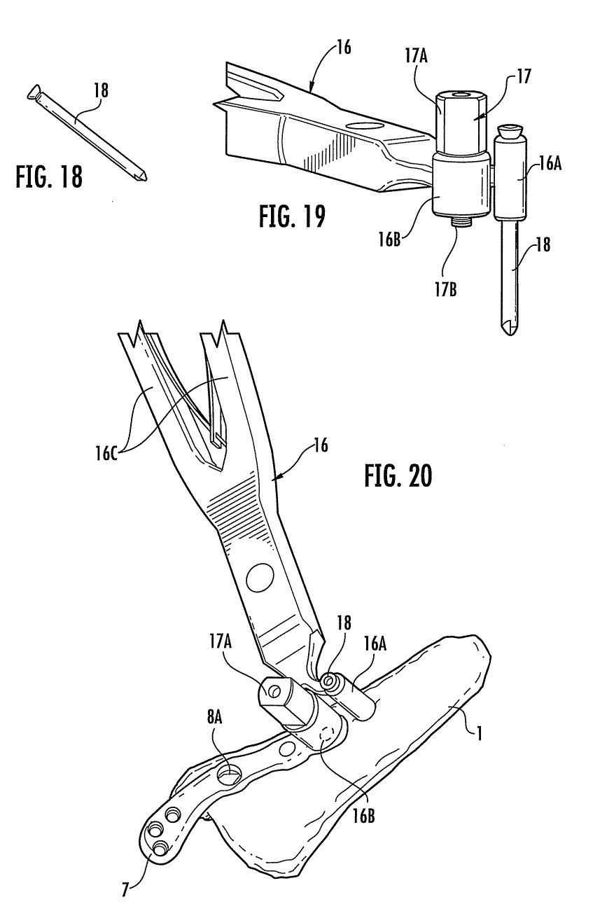

Advantageously, and as can be seen more particularly in FIGS. 10 and 11, the antero-medial plate 7 (preferably like the antero-lateral plate 3) can have, as seen in profile, substantially the shape of a spoon, the tibio-medial strip 8 corresponding to the handle of the spoon, while the link zone 10 and the talo-medial tab 9 can form the concave portion (receptacle) of the spoon. Advantageously, the talo-medial tab 9 can extend in a main longitudinal direction X-X′ that can be inclined laterally, i.e. towards the outside of the body of the patient, relative to the main longitudinal direction Y-Y′ in which the tibio-medial strip 8 extends. In other words, as seen from the front (FIG. 8), the antero-medial plate 7 can have a main body that can be substantially rectilinear, corresponding substantially to the tibio-medial strip 8 and to the link zone 10, which main body is provided with a spatulate element that forms a non-zero angle with the main body, the spatulate element in question corresponding to the talo-medial tab 9.

The antero-lateral plate 3 can have a shape that can be substantially symmetrical, about the anterior tibial crest 1B, to the shape of the antero-medial plate 7, i.e. the talo-lateral tab 5 can be advantageously inclined towards the inside of the body of the patient relative to the tibio-lateral strip 4.

By this feature, the tabs 5, 9 can converge towards the middle of the neck 2A of the talus 2, thereby making it possible to take advantage of a bone surface that is not only effective for fastening (because it has the characteristic of being generally flat in that region) but that is also easy for the surgeon to reach and to identify.

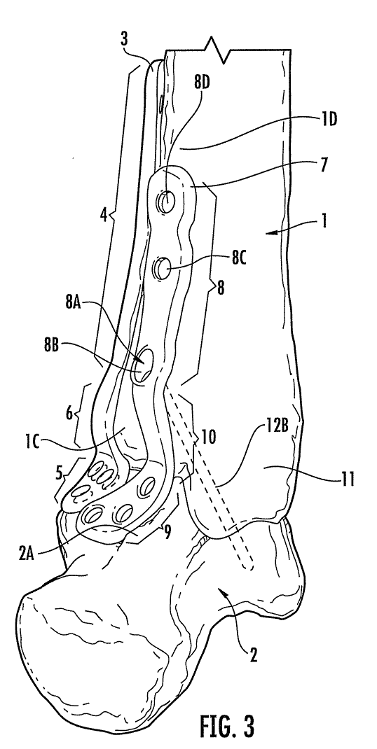

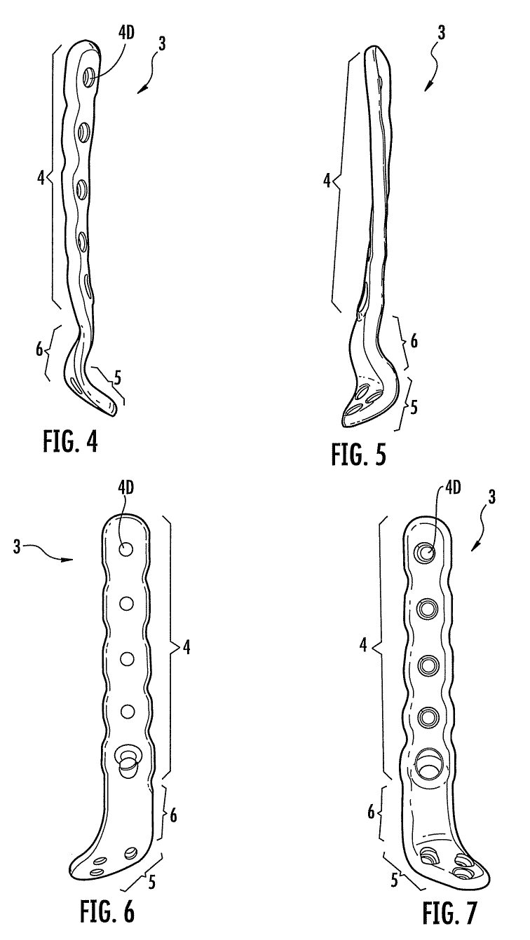

Advantageously, the systems and methods of the present disclosure further can comprise at least one first anchor member 12 that can be provided with a head 12A and with a bone-anchor shank 12B that can extend from the head 12A, the tibio-medial strip 8 being provided with at least a first orifice 8A provided with a first bearing surface 8B against which the head 12A is designed to come into abutment, the first bearing surface 8B advantageously being shaped so that the anchor shank 12B can extend in a direction enabling it to pass through both the tibia 1 and the talus 2 (as shown in dashed lines in FIG. 3), the shank 12B thus forming a trans-articular anchor shank. In another particularly advantageous embodiment, the first bearing surface 8B of the first orifice 6A can advantageously be shaped so that the anchor shank 12B can extend through the tibia 1, through the talus 2, and through the calcaneum (not shown). Thus, by combining an adapted orientation of the first bearing surface 8B of the first orifice 8A and a sufficient length for the shank 12B of the corresponding first anchor member 12, it is possible to achieve trans-articular fastening that secures together the antero-medial plate 7, the tibia 1, the talus 2, and optionally the calcaneum.

The first anchor member 12 can be constituted by a bone screw designed to be screwed both into the tibia 1 and into the talus 2, and its length can thus be appropriate for this purpose.

Advantageously, as shown in the figures, the tibio-medial strip 8 can be provided with a plurality of orifices 8A, 8C, 8D, each of which can be designed to be associated with a corresponding anchor member. Advantageously, the first orifice 8A can be positioned as close as possible to the link zone 10 of the tibio-medial strip 8, i.e. it can constitute the orifice that is furthest away from the tibio-medial strip 8. In other words, the orifice that is closest to the link zone 10 can correspond to the first orifice 8A.

Thus, in the example shown in the figures, the tibio-medial strip 8 can be provided with three orifices 8A, 8C, 8D, namely a proximal orifice 8D, an intermediate orifice 8C, and a distal orifice that corresponds to the first orifice 8A.

In order to facilitate positioning the shank 12B of the first anchor member 12 forming the trans-articular fastening member, the first orifice 8A can be positioned on the tibio-medial strip 8 in a manner such as to find itself substantially in the vicinity of the beginning of the bulge of bone formed by the pilon 1C. This position is particularly conducive to easily obtaining stable and robust fastening by transpiercing the tibia 1 and the talus 2 with the same shank 12B.

In order to facilitate fitting the first trans-articular anchor member 12, the first bearing surface 8B of the first orifice 8A advantageously can extend in a midplane P disposed slantingly relative to the main longitudinal direction Y-Y′ in which the tibio-medial strip 8 extends. In other words, and as shown in the figures, the first orifice 8A can extend through the thickness of the tibio-medial strip 8 between the inside face 13 of the strip 8 that is designed to find itself in contact with the bone and the opposite outside face 14, the first bearing surface 8B being disposed slantingly relative to the inside face 13 and/or to the outside face 14.

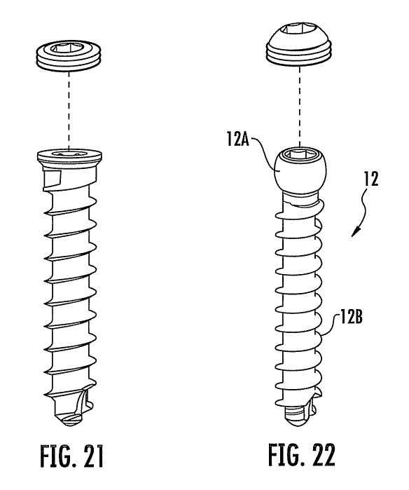

The first bearing surface 8B of the first orifice 8A can have a substantially rounded shape, which can be in the shape of a sphere portion, so as to enable the orientation of the first anchor member 12 relative to the tibio-medial strip 8 to be adjusted. For this purpose, the head 12A of the first anchor member 12, or at least the portion of the head 12A that is designed to come into contact with the first bearing surface 8B, can have a complementary spherical shape (cf. FIG. 22).

The surgeon can thus finely adjust the orientation of the first anchor member 12 relative to the tibio-medial strip 8 so as to make sure, e.g. by X-ray checking, that the shank 12B does indeed extend both through the tibia 1 and through the talus 2.

Preferably, in order to facilitate good fastening to the bone, the shank 12B of the first anchor member 12 can have a diameter (e.g. a diameter of 3.5 millimeters (mm)) that can be significantly greater than the diameter (e.g. 3 mm) of the shanks of the other anchor members used in co-operation with the other orifices 8C, 8D provided through the tibio-medial strip 6.

Advantageously, the talo-medial tab 9 can also be provided with orifices, such as with three such orifices that can be disposed in a triangle configuration, the talo-medial tab advantageously presenting a shape that is wider than the shape of the tibio-medial strip 8 and than the shape of the link zone 10. The link zone 10 can be provided without any orifices, thereby facilitating any plate-folding steps that the surgeon might wish to perform during the operation, so as to adapt to accommodate specific anatomical conditions.

The antero-medial plate 7 can thus be provided with three fastening orifices in line that are provided in its tibio-medial strip 8, and with three fastening orifices not in line that are provided in its talo-medial tab 9.

As explained above, the first orifice 8A advantageously can have a spherical bearing surface 8B making it possible to adjust the angulation of the screw with which it is designed to co-operate, the spherical bearing surface 8B further being oriented specifically to direct the screw both towards the tibia 1 and towards the talus 2, the screw in question (which corresponds to the first anchor member 12) thus forming a trans-articular screw.

Preferably, each of the other orifices can be provided with a frustoconical bearing surface surmounted by an inside thread designed to receive a lock screw making it possible to lock the screw head (system of the Surfix® type described in particular in French Patent Application FR-07 04449 filed on Jun. 21, 2007, and in European Patent Application EP-0 345 133 filed on May 26, 1989, the contents of those applications being incorporated into the present description).

Advantageously, as described in above-mentioned Patent Application FR-07 04449, each orifice provided in the antero-medial plate 7, preferably except for the first orifice 8A, can be used equally well either with a screw having a frustoconical head as shown in FIG. 21 (for particularly firm and stable fastening, or with a screw having a spherical head as shown in FIG. 22 (for fastening making it possible to adjust the angulation of the screw relative to the plate), a lock nut always being used, making it possible to secure the screws effectively to the plate.

The antero-lateral plate 3 can be substantially similar to the design of the antero-medial plate 7, except that the tibio-lateral strip 4 can be longer than the tibio-medial strip 8, and can be provided with more orifices (five orifices, each of which is designed to receive a fastener member, of the screw type, the distal orifice, situated closest to the link zone 6, having a bearing surface disposed slantingly so as to enable a screw 15 that is longer and of larger diameter than the other screws to pass both through the tibia 1 and through the talus 2.

Advantageously, the systems and methods of the present disclosure further can comprise a compression tool 16 for compressing the joint between the tibia 1 and the talus 2. The compression tool 16 can comprise: a first jaw 16A that can be fastened temporarily to the tibia 1; a second jaw 16B that can be fastened temporarily to either one of the plates, namely the antero-lateral plate 3 or the antero-medial plate 7; and a closure device (preferably a manual device) for bringing the first and second jaws 16A, 16B closer together.

One or both of the antero-lateral plate 3 and the antero-medial plate 7 can be provided with a tapped hole into which the second jaw 16B itself or an independent piece 17 mounted on the second jaw 16B is designed to be screwed. Advantageously, the tapped hole can be provided in the tibio-lateral strip 4 or in the tibio-medial strip 8 (preferably in the tibio-lateral strip 4) and even more preferably towards the free end of the strip in question. As shown in the figures, the compression tool 16 can be in the form of a pair of pliers, the second jaw 16B being provided with a sleeve into which a bush 17A is engaged, which bush is provided with an outside thread 17B, the outside thread 17B preferably being designed to be screwed either into the inside thread of the proximal orifice 4D provided in the tibio-lateral strip 4, closest to the free end thereof, or into the inside thread of the proximal orifice 8D provided in the tibio-medial strip 8, closest to the free end thereof (as shown in FIG. 20). The second jaw can thus be fastened to the tibio-lateral strip 4 or to the tibio-medial strip 8 by being screwed into the orifice 4D, 8D in question. The tapping of the orifice in question can thus perform two functions because it makes it possible firstly to secure the second jaw 16B of the compression tool 16 to the plate in question, and secondly, once the compression has been performed, to co-operate with a lock screw to lock the head of the fastener screw inserted into the orifice in question (SUREFIX® system described in above-mentioned Applications FR-07 04449 and EP-0 345 133).

Naturally, the present disclosure also relates independently to the compression tool in question per se, and similarly relates independently to the antero-lateral plate 3 on its own and to the antero-medial plate 7 on its own.

In addition to the disclosure above, each of the plates described above and shown in the drawings individually constitutes inventive subject matter, and it is envisioned according to the present disclosure that each plate can be utilized in an individual capacity.

One example of a method of using a system of the present disclosure is described below.

Firstly, the patient is positioned in the supine position on an X-ray operating table. A longitudinal anterior incision of in the range 10 centimeters (cm) to 12 cm is formed laterally directly in the anterior tendon of the tibia. The surgeon then performs surgical cleaning in order to uncover both the anterior face of the tibia 1 and the anterior face of the talus 2. In particular, the surgeon removes the cartilage from the dome of the talus and from the tibial plafond. Bone grafts can be inserted into the talo-tibial joint.

The surgeon then fits the antero-lateral plate 3 preferably first. For this purpose, the surgeon first fastens the talo-lateral tab 5 to the neck 2A of the talus 2, such as by using locking fasteners, such as three screw and lock-screw sets of the SUREFIX® type, as shown in FIGS. 21 and 22. The talo-lateral tab 5 is positioned on the lateral side of the neck of the talus 2, and is fastened by means of three screws that are inserted into respective ones of the three fastener orifices provided in the talo-lateral tab 5.

The surgeon then compresses the joint between the tibia 1 and the talus 2.

For this purpose, the first jaw 16A of the pliers constituting the compression tool 16 is fastened to the tibia 1, by means of a temporary screw 18, while the second jaw 16B is screwed, via the bush 17A, into the tibia 1, in alignment with the fastening orifices provided in the tibio-lateral strip 4.

The compression pliers are thus fastened, in the open position, both to the tibio-lateral strip 4 and to the talus 2. The surgeon then exerts a force on the arms 16C of the pliers, thereby causing the first and second jaws 16A, 16B to move closer together. More precisely, the first jaw 16A remains stationary while the second jaw 16B moves towards the first jaw 16A, entraining with it upwards the antero-lateral plate 3 and the talus 2 to which the plate 3 is fastened. The talus 2 is thus compressed against the distal base of the tibia 1. With the joint being held thus in compression, the tibio-lateral strip 4 is fastened permanently to the tibia 1 by means of screws inserted into the orifices provided in the tibio-lateral strip 4.

Once the antero-lateral plate 3 is fastened in this way, the surgeon inserts and fastens the antero-medial plate 7 in a similar manner.

What is claimed is:

1. A system for immobilizing the joint between the tibia and the talus of a human patient, the system comprising:
an antero-lateral plate secured to an anterior face of the tibia and the talus, the antero-lateral plate including a tibio-lateral strip and a talo-lateral tab interconnected by a link zone; and
an antero-medial plate secured to an anterior face of the tibia and the talus, the antero-medial plate including a tibio-medial strip and a talo-medial tab interconnected by a link zone.

2. A system according to claim 1, wherein the antero-lateral plate is shaped so that at least one portion of the tibio-lateral strip rests substantially against the anterior lateral face of the tibia that is situated towards the outside of the body of the patient relative to the anterior tibial crest and the talo-lateral tab rests substantially against the talus.

3. A system according to claim 2, wherein the antero-lateral plate has a shape that is significantly twisted so as to match the outline of the anterior side face of the tibia and the outline of the neck of the talus

4. A system according to claim 1, wherein the antero-medial plate is shaped so that at least one portion of the tibio-medial strip rests substantially against the anterior medial face of the tibia that is situated towards the inside of the body of the patient relative to the anterior tibial crest and the talo-medial tab rests substantially against the talus.

5. A system according to claim 1, wherein the link zone of the antero-medial plate is designed to come into register with at least a portion of the tibial pilon, the link zone having a shape that is substantially complementary to the shape of the portion of the tibial pilon.

6. A system according to claim 1, wherein the antero-medial plate has a shape that is significantly twisted so as to match the outline of the anterior medial face of the tibia and the outline of the neck of the talus;

7. A system according to claim 1, wherein the tibio-medial strip has a shape that is substantially arcuate so as to match both the outline of the anterior medial face of the tibia and the outline of at least one portion of the tibial pilon.

8. A system according to claim 1, wherein the antero-medial plate has, as seen in profile, substantially the shape of a spoon.

9. A system according to claim 1, wherein the talo-medial tab extends in a main longitudinal direction that is inclined towards the outside of the body of the patient relative to a main longitudinal direction in which the tibio-medial strip extends.

10. A system according to claim 1, further comprising at least a first anchor member provided with a head and with a bone anchor shank that extends from the head, the tibio-medial strip being provided with at least a first orifice provided with a first bearing surface against which the head is designed to come into abutment, the bearing surface being shaped so that the anchor shank can extend in a direction enabling it to pass both through the tibia and through the talus.

11. A system according to claim 10, wherein the first bearing surface extends in a midplane disposed slantingly relative to a main longitudinal direction in which the tibio-medial strip extends.

12. A system according to claim 10, wherein the first bearing surface has a substantially rounded shape so as to make it possible to adjust the orientation of the first anchor member relative to the tibio-medial strip.

13. A system according to claim 10, wherein the tibio-medial strip is provided with a plurality of orifices, each of which is designed to be associated with a corresponding anchor member, the orifice that is closest to the link zone of the antero-medial plate corresponding to the first orifice.

14. A system according to claim 13, wherein the shank of the first anchor member has a diameter that is significantly grater than the diameter of the shanks of the other anchor members.

15. A system according to claim 1, further comprising compression tool for compressing the joint between the tibia and the talus, the compression tool comprising:
a first jaw configured to be fastened temporarily to the tibia;
a second jaw configured to be fastened temporarily to one of the antero-lateral plate or to the antero-medial plate; and
a closure device configured to bring the first and second jaws closer together.

16. A system according to claim 15, wherein one or both of the antero-lateral plate and the antero-medial plate is/are provided with a tapped hole into which either the second jaw itself or a piece mounted on the second jaw is configured to be screwed.

17. A system according to claim 15, wherein the tapped hole is provided in the tibio-lateral strip or in the tibio-medial strip, preferably towards the free end of the strip.

18. An antero-lateral plate for immobilizing the joint between the tibia and the talus of a human patient, the antero-lateral plate having an anatomical shape that substantially matches the shape of portions of the tibia and the talus of the human patient that are situated towards the outside of the body of the patient relative to the anterior tibial crest.

19. An antero-lateral plate for use in a system for immobilizing the joint between the tibia and the talus of a human patient, the antero-lateral plate comprising:
a tibio-lateral strip having an elongated shape, wherein at least one portion of the tibio-lateral strip rests substantially against the anterior lateral face of the tibia that is situated towards the outside of the body of the patient relative to the anterior tibial crest;
a talo-lateral tab having a shape such that the talo-lateral tab rests substantially against the neck of the talus; and
a link zone interconnecting the tibio-lateral strip to the talo-lateral tab, the link zone having a curved shape so as to match the outline of the tibial pilon.

20. An antero-medial plate for use in a system for immobilizing the joint between the tibia and the talus of a human patient, the antero-medial plate having an anatomical shape that substantially matches the shape of portions of the tibia and the talus of the human patient that are situated towards the inside of the body of the patient relative to the anterior tibial crest.

21. An antero-medial plate for use in a system for immobilizing the joint between the tibia and the talus of a human patient, the antero-medial plate comprising;
a tibio-medial strip having a shape such that at least one portion of the tibio-medial strip rests substantially against the anterior medial face of the tibia that is situated towards the inside of the body of the patient relative to the anterior tibial crest;
a talo-medial tab having a shape such that the talo-medial tab rests substantially against the neck of the talus; and
a link zone interconnecting the tibio-medial strip to the talo-medial tab, the link zone having a curved shape so as to match the outline of the tibial pilon.

22. A system for immobilizing the joint between the tibia and the talus of a human patient, the system comprising:
an antero-lateral plate, itself comprising a tibio-lateral strip and a talo-lateral tab interconnected by a link zone, the tibio-lateral strip, the talo-lateral tab, and the link zone being shaped so that at least one portion of the tibio-lateral strip rests substantially against the anterior lateral face of the tibia that is situated towards the outside of the body of the patient relative to the anterior tibial crest and the talo-lateral tab rests substantially against the talus; and
an antero-medial plate, itself comprising a tibio-medial strip and a talo-medial tab interconnected by a link zone, the tibio-medial strip, the talo-medial tab, and the link zone being shaped so that at least one portion of the tibio-medial strip rests substantially against the anterior medial face of the tibia that is situated towards the inside of the body of the patient relative to the anterior tibial crest and the talo-medial tab rests substantially against the talus.

23. A method for immobilizing the joint between the tibia and the talus of a human patient, the method comprising:
creating a longitudinal anterior incision in an anterior tendon of the tibia of the patient;
exposing the distal tibia and the neck of the talus;
implanting an antero-lateral plate against an anterior side face of the tibia that is situated towards the outside of the body of the patient relative to the anterior tibial crest; and
implanting an antero-medial plate against an anterior medial face of the tibia that is situated towards the inside of the body of the patient relative to the anterior tibial crest.

24. A method according to claim 23, wherein implanting the antero-lateral plate comprises:
positioning a plate having a talo-lateral tab and a tibio-lateral strip against an anterior side face of the tibia;
fastening the talo-lateral tab to the neck of the talus; and
fastening the tibio-lateral strip to the anterior lateral face of the tibia.

25. A method according to claim 24, wherein implanting the antero-lateral plate further comprises compressing the joint between the tibia and the talus between the steps of fastening the talo-lateral tab to the neck of the talus and fastening the tibio-lateral strip to the anterior lateral face of the tibia, the compressing comprising:
temporarily fastening a first jaw of a compression tool to the tibia;
temporarily fastening a second jaw of the compression tool to the antero-lateral plate; and
exerting a force on the compression tool to move the first jaw and the second jaw of the compression tool closer together.

26. A method according to claim 23, wherein implanting an antero-medial plate comprises implanting a plate having a shape that is significantly twisted so as to match the outline of the anterior medial face of the tibia and the outline of the neck of the talus.

27. A method according to claim 23, wherein implanting an antero-medial plate comprises:
positioning a plate having a talo-medial tab and a tibio-medial strip against an anterior medial face of the tibia;
fastening the talo-medial tab to the neck of the talus; and
fastening the tibio-medial strip to the anterior medial face of the tibia.

28. A method according to claim 27, wherein implanting the antero-medial plate further comprises compressing the joint between the tibia and the talus between the steps of fastening the talo-medial tab to the neck of the talus and fastening the tibio-medial strip to the anterior medial face of the tibia, the compressing comprising:
temporarily fastening a first jaw of a compression tool to the tibia;
temporarily fastening a second jaw of the compression tool to the antero-medial plate; and
exerting a force on the compression tool to move the first jaw and the second jaw of the compression tool closer together.
